(19) United States
(12) Patent Application Publication
(10) Pub. No.: US 2002/0137702 A1
(43) Pub. Date: Sep. 26, 2002

(54) Yeast receptor

(76) Inventors: Robert Alan Arkowitz, Nice (FR); Peter Michael Aljoscha Nern, Los Angeles, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/732,180

(22) Filed: Dec. 7, 2000

Related U.S. Application Data
(60) Provisional application No. 60/169,699, filed on Dec. 7, 1999.

(51) Int. Cl. A61K 48/00; C07H 21/04; A61K 38/16; C12P 21/02

(52) U.S. Cl. 514/44; 514/12; 536/23.7; 435/69.1; 530/350

(57) ABSTRACT

A nucleotide sequence is described. The nucleotide sequence or the expression product of the nucleotide sequence has the capability of not substantially affecting the interaction of Gβ with a Cdc24p obtainable from C. albicans or a homologue thereof that is usually capable of being associated with the Cdc24p obtainable from C. albicans or the homologue thereof

The present invention relates to nucleotide sequences and protein sequences. In particular, the present invention relates to nucleotide sequences and protein sequences that affect interactions of cellular components.

According to Cerione and Zheng (The Dbl family of oncogenes Current Opinion In Cell Biology 8, 216-222 (1996)), genetic screening and biochemical studies during the past years have led to the discovery of a certain family of cell growth regulatory proteins and oncogene products for which the Dbl oncoprotein is the prototype. Another review on Dbl is presented by Machesky and Hall (1996 Trends In Cell Biology 6 pp 3-4-310).

Cerione and Zheng (ibid) say that proto-Dbl is a 115 kDa cytoskeleton-associated protein that is found in tissues such as brain, ovary, testis and adrenal glands. Oncogenic activation of proto-Dbl occurs as a result of an amino-terminal truncation of proto-Dbl which leaves residues 498-925 fused with the product of an as yet unidentified gene which is localised on chromosome 3.

Cerione and Zheng also say that a region located between residues 498 and 674 of proto-Dbl—which is retained by oncogenic Dbl—has significant similarities with the Saccharomyces cerevisiae cell division cycle molecule Cdc24p and the breakpoint cluster gene product Bcr (see also Hart et al 1991 Nature 354 311-314; Miyamoto et al 1991 Biochem Biophys Res Commun 181 604-610; Ron et al 1991 New Biol 3 372-379). This region—which is referred to as being the DH domain—was later shown to be responsible for the GEF (GDP-GTP Exchange Factor—otherwise known as a guanine nucleotide exchange factor) activity of the Dbl oncoprotein and to be critical for its transforming function (see also Hart et al J Biol Chem 269 62-65).

Cerione and Zheng also report that since the initial identification of Dbl as a GEF for Rho-type GTP binding proteins, a number of oncogene products and growth regulatory molecules have been shown to contain a DH domain in tandem with another region designated PH (i.e. a pleckstrin homology domain which is found between residues 703-812 in of proto-Dbl). Many of these products and molecules, such as Bcr, Cdc24, Sos, Vav, ect-2, Ost, Tim, Lbc, Lfc and Dbc, form a family of GEFs which have been implicated in cell growth regulation. Cerione and Zheng provide details on each of these products and molecules. In addition, these and other products and molecules are discussed below.

Cerione and Zheng (ibid) end their Abstract by saying:

“Despite the increasing interest in the Dbl family of proteins, there is still a good deal to learn regarding the biochemical mechanisms that underlie their diverse biological functions.”

As mentioned above, it is known that proto-Dbl has significant similarities with the S. cerevisiae cell division cycle molecule Cdc24p which is a GEF for the Rho-family GTPase molecule Cdc42p (see again Hart et al 1991 Nature 354 311-314; Miyamoto et al 1991 Biochem Biophys Res Commun 181 604-610. Ron et al 1991 New Biol 3 372-379; Zheng et al 1994 J Biol Chem 269 2369-2372). However, whilst it is known that the Rho-family GTPases and their regulators are essential for cytoskeletal reorganisation and transcriptional activation in response to extracellular signals^{1,2}, little is known about what links these molecules to membrane receptors. For example, in the budding yeast S. cerevisiae, haploid cells respond to mating pheromone through a G-protein coupled receptor (Ste2p/Ste3p) via Gβγ (Ste4p/Ste18p) resulting in cell cycle arrest, transcriptional activation, and polarised growth towards a mating partner^{4,5}. Recently, the Rho-family GTPase Cdc42p and its exchange factor Cdc24p have been implicated in the mating process^{6,7} but their specific role is unknown.

In our studies on S. Cerevisiae, which are outlined in WO 99/18213, we have been able to identify hitherto unrecognised regions that play a key role in the interaction of cellular components. By way of example, we have identified novel cdc24 alleles which do not affect vegetative growth but drastically reduce the ability of yeast cells to mate. When exposed to mating pheromone these mutants arrest growth, activate transcription, and undergo characteristic morphological and actin cytoskeleton polarisation. However, the mutants are unable to orient towards a pheromone gradient and instead position their mating projection adjacent to their previous bud site. Strikingly, these mutants are specifically defective in the binding of Cdc24p to Gβγ. This work demonstrates that the association of a GEF and the βγ-subunit of a hetero-trimeric G-protein (Gβγ) links receptor-mediated activation to oriented cell growth.

This finding has broad implications—not only for the design of anti-fungal drugs, such as those that could be directed against the yeast Candida, but also in the screening and design of agents that can affect oncogenes such as Dbl, in particular proto-Dbl.

However, a complexity of working with Candida species, such as C. albicans, is that the organism is diploid and in a number of cases, the two alleles in the diploid organism have diverged resulting in alleles with different and/or non-identical function. By way of example, an academic consortium accessible at http://alces.mediumn.edu/Candida.html have annotated, from Blast similarity searches, some small portions of the C. albicans gene (CDC24) which encodes the Cdc24 protein (Cdc24p). Using a shotgun procedure, this academic consortium has only identified small portions of the CDC24 gene encoding Cdc24p and these portions have only been annotated as CDC24 because they pick up the S. cerevisiae CDC24 in a BLAST search. However, the intact Candida gene encoding CDC24 has not been annotated as a considerable number of the regions of the C. albicans CDC24 do not line up well with S. cerevisiae CDC24.

Thus, the present invention seeks to overcome the problems associated with the cloning and characterisation of the CDC24 gene obtainable from C. albicans.

Thus, according to one broad aspect of the present invention there is provided a GDP-GTP Exchange Factor (GEF) obtainable from C. albicans wherein the GEF is Cdc24p and wherein the Cdc24p GEF is capable of interacting with proteins such as Gβ. These interactions are likely to be necessary for polarised cell growth and hence are appropriate anti-fungal targets.

These and other aspects of the present invention are set out in the claims. By way of example, in a broad aspect, the present invention provides a nucleotide sequence shown as SEQ I.D. No. 1 or a derivative, fragment, variant or homologue thereof, wherein the expression product of the nucleotide sequence has the capability of not substantially affecting the interaction of proteins such as Gβ with a GEF or a homologue thereof that is usually capable of being associated therewith.

As used herein, the term “Gβ” includes Gβ and any Gβ associated protein such as Ste4p/Ste18p and/or a Rho-family GTPase (such as Cdc42p).

The term “expression product of the nucleotide sequence has the capability of not substantially affecting the interaction of proteins such as Gβ with GEF or a homologue thereof that is usually capable of being associated therewith” means that if the expression product were to be present within the GEF and the GEF were to be contacted with proteins such as Gβ then the expression product would not substantially affect the interaction of proteins such as Gβ with the GEF.

Thus, alternatively expressed, the present invention covers a nucleotide sequence shown as SEQ I.D. No. 1 or a derivative, fragment, variant or homologue thereof, wherein the expression product of the nucleotide sequence has the capability of not substantially affecting the interaction of proteins such as Gβ with a GEF or a homologue thereof that is usually capable of being associated therewith if the expression product were to be present within the GEF and the GEF were to be contacted with proteins such as Gβ.

With this aspect of the present invention, the expression product need not necessarily be present within the GEF and/or the GEF need not necessarily be contacted with proteins such as Gβ. By way of example, the expression product can be part of a truncated GEF and/or part of a fused protein. However, if the expression product were present within GEF, then preferably the GEF is not in its natural environment. By way of example, the GEF can be in an isolated form—such as in an assay device, Likewise, if the expression product were contacted with proteins such as Gβ then preferably the proteins such as Gβ is not in its natural environment. By way of example, the proteins such as Gβ can be in an isolated form—such as in an assay device.

The present invention also covers a mutant of the nucleotide sequence shown as SEQ I.D. No. 1 or a derivative, fragment, variant or homologue thereof, wherein the expression product of the mutant nucleotide sequence has the capability of substantially affecting the interaction of proteins such as Gβ with a GEF or a homologue thereof that is usually capable of being associated therewith.

The term “expression product of the mutant nucleotide sequence has the capability of substantially affecting the interaction of proteins such as Gβ with a GEF or a homologue thereof that is usually capable of being associated therewith” means that if the expression product were to be present within a GEF like entity (such as GEF bearing that mutation) and that GEF like entity were to be contacted with Gβ then the expression product would substantially affect the interaction of proteins such as Gβ with that GEF like entity.

Thus, alternatively expressed, the present invention also covers a mutant of the nucleotide sequence shown as SEQ I.D. No. 1 or a derivative, fragment, variant or homologue thereof, wherein the expression product of the mutant nucleotide sequence has the capability of substantially affecting the interaction of proteins such as Gβ with a GEF or a homologue thereof that is usually capable of being associated therewith if the expression product were to be present within GEF and the GEF were to be contacted with proteins such as Gβ

With this aspect of the present invention, the expression product need not necessarily be present within the GEF like entity and/or the GEF like entity need not necessarily be contacted with proteins such as Gβ. By way of example, the expression product can be part of a truncated GEF and/or part of a fused protein. The GEF like entity may be in an isolated form—such as in an assay device. Likewise,. if the expression product were contacted with proteins such as Gβ then preferably the proteins such as Gβ is not in its natural environment. By way of example, the Gβ can be in an isolated form—such as in an assay device.

The present invention also covers in a broad aspect a nucleotide sequence shown as SEQ I.D. No. 1 or a derivative, fragment, variant or homologue thereof, wherein the expression product of the nucleotide sequence has the capability of not substantially affecting the interaction of Gβ with Cdc24p or a homologue thereof that is usually capable of being associated therewith.

The term “expression product of the nucleotide sequence has the capability of not substantially affecting the interaction of Gβ with Cdc24p or a homologue thereof that is usually capable of being associated therewith” means that if the expression product were to be present within Cdc24p and the Cdc24p were to be contacted with Gβ then the expression product would not substantially affecting the interaction of Gβ with Cdc24p.

Thus, alternatively expressed, the present invention covers in a broad aspect a nucleotide sequence shown as SEQ l.D. No. 1 or a derivative, fragment, variant or homologue thereof, wherein the expression product of the nucleotide sequence has the capability of not substantially affecting the interaction of Gβ with Cdc24p or a homologue thereof that is usually capable of being associated therewith if the expression product were to be present within Cdc24p and the Cdc24p were to be contacted with Gβ.

With his aspect of the present invention, the expression product need not necessarily be present within Cdc24p and/or the Cdc24p need not necessarily be contacted with Gβ. By way of example, the expression product can be part of a truncated Cdc24p and/or part of a fused protein. However, if the expression product is present within Cdc24p, then preferably the Cdc24p is not in its natural environment. By way of example, the Cdc24p can be in an isolated form—such as in an assay device. Likewise, if the expression product were contacted with Gβ then preferably the Gβ is not in its natural environment. By way of example, the Gβ can be in an isolated form—such as in an assay device.

By way of further example, the present invention also covers a mutant of the nucleotide sequence shown as SEQ I.D. No.1 or a derivative, fragment, variant or homologue thereof, wherein the expression product of the mutant nucleotide sequence has the capability of substantially affecting the interaction of Gβ with Cdc24p or a homologue thereof that is usually capable of being associated therewith.

The term “expression product of the mutant nucleotide sequence has the capability of substantially affecting the interaction of Gβ with Cdc24p or a homologue thereof that is usually capable of being associated therewith” means that if the expression product were to be present within a Cdc24p like entity (such as Cdc24p bearing that mutation) and that Cdc24p like entity were to be contacted with Gβ then the expression product would substantially affect the interaction of Gβ with that Cdc24p like entity.

With this aspect of the present invention, the expression product need not necessarily be present within the Cdc24p like entity and/or the Cdc24p like entity need not necessarily be contacted with Gβ. By way of example, the expression product can be part of a truncated Cdc24p and/or part of a fused protein. The Cdc24p like entity may be in an isolated form—such as in an assay device. Likewise, if the expression product were contacted with Gβ then preferably the Gβ is not in its natural environment. By way of example, the Gβ can be in an isolated form—such as in an assay device.

In a preferred aspect, the present invention covers the sequences of the present invention in isolated form—in other words the sequences are not in their natural environment and when they have been expressed by their natural coding sequences which are under the control of their natural expression regulatory elements (such as the natural promoter etc.). By way of example the sequences may be in an assay device.

It is to be noted that the nucleotide sequence presented as SEQ ID No. 1 is quite different to the DH domain and the PH domain discussed by Cerione and Zheng (ibid). It is also to be noted that the nucleotide sequence presented as SEQ ID No. 1 covers regions in addition to the DH domain and the PH domain.

The nucleotide sequence (SEQ ID No 1) and its expression product (SEQ ID No 2) may affect the interaction of C. albicans Cdc24p with a β subunit (such as Ste4p) or even a βγ subunit (such as Ste4p/Ste18p) of a hetero-trimeric G-protein (herein referred to as “Gβ”). If the interaction is detrimentally affected (such as lost) then his may in turn prevent (or at least reduce) signalling (possibly GEF activity) being passed to the the Rho-family GTPase (Cdc42p). Hence, the present invention also covers the use of any one or more of the aforementioned aspects of the present invention to have an effect on a signal being passed to the Rho-family GTPases.

The term “derivative, fragment, variant or homologue” in relation to the nucleotide Sequence ID No. 1 of the present invention includes any substitution of, modification of, replacement of, deletion of or addition of one (or more) nucleic acid from or to the sequence providing the resultant nucleotide sequence or the expression product thereof has the capability of not substantially affecting the interaction of Gβ with a Cdc24p obtainable from C. albicans or a homologue thereof that is usually capable of being associated with a Cdc24p obtainable from C. albicans or the homologue thereof. In particular, the term “homologue” covers homology with respect to function. With respect to sequence homology (i.e. similarity), preferably there is at least 75%, more preferably at least 85%, more preferably at least 90% homology to the sequence shown as SEQ ID No.1 in the attached sequence listings. More preferably there is at least 95%, such as at least 98%, homology to the sequence shown as SEQ ID No. 1 in the attached sequence listings.

The term “derivative, fragment, variant or homologue” in relation to the protein Sequence ID) No. 2 of the present invention includes any substitution of, modification of, replacement of, deletion of or addition of one (or more) amino acid from or to the sequence providing the resultant amino acid sequence has the capability of not substantially affecting the interaction of Gβ with a Cdc24p obtainable from C. albicans or a homologue thereof that is usually capable of being associated with a Cdc24p obtainable from C. albicans or the homologue thereof. In particular, the term “homologue” covers homology with respect to function. With respect to sequence homology (i.e. similarity), preferably there is at least 75%, more preferably at least 85%, more preferably at least 90% homology to the sequence shown as SEQ ID No.2 in the attached sequence listings. More preferably there is al least 95%, such as at least 98%, homology to the sequence shown as SEQ ID No. 2 in the attached sequence listings.

In particular, the term “homology” as used herein may be equated with the term “identity”. Relative sequence homology (i.e. sequence identity) can be determined by commercially available computer programs that can calculate % homology between two or more sequences. Typical examples of such computer programs are BLAST and CLUSTAL.

Sequence homology (or identity) may moreover be determined using any suitable homology algorithm, using for example default parameters. Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (see http://www.ncbi.nih.gov/BLAST/blast_help.html) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al (1994) Nature Genetics 6:119-129.

The five BLAST programs available at http://www.ncbi.nlm.nih.gov perform the following tasks:

blastp compares an amino acid query sequence against a protein sequence database;

blastn compares a nucleotide query sequence against a nucleotide sequence database;

blastx compares the six-frame conceptual translation products of a nucleotide query sequence (both strands) against a protein sequence database;

tblastn compares a protein query sequence against a nucleotide sequence database dynamically translated in all six reading frames (both strands).

tblastx compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRIPTIOnucleotide sequence Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Mltschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Preferably, sequence comparisons are conducted using the simple BLAST search algorithm provided at http://www.ncbi.nlm.nih.gov/BLAST.

More preferably, sequence comparisons are conducted using the simple BLAST 2 search algorithm provided at http://wvww.ncbi.nlm.nih.gov/gorf/wblast2.cgi.

Other computer program methods to determine identify and similarity between the two sequences include but are not limited to the GCG program package (Devereux et al 1984 Nucleic Acids Research 12:387and FASTA (Atschul et al 1990 J Molec Biol 403-410).

The term “variant” also encompasses sequences that are complementary to sequences that are capable of hydridising to the nucleotide sequences presented herein.

Preferably, the term “variant” encompasses sequences that are complementary to sequences that are capable of hydridising under stringent conditions (eg. 65° C. and 0.1×SSC {1×SSC=0.15 M NaCl, 0.015 Na₃ citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

The term “hybridization” as used herein shall include “the process by which a strand of nucleic acid joins with a complementary strand through base pairing” (Coombs J (1994) Dictionary of Biotechnology, Stockton Press, New York N.Y.) as well as the process of amplification as carried out in polymerase chain reaction technologies as described in Dieffenbach C W and G S Dveksler (1995, PCR Primer, a Laboratory Manual, Cold Spring Harbor Press, Plainview N.Y.).

Also included within the scope of the present invention are polynucleotide sequences that are capable of hybridizing to the nucleotide sequence of the present invention or other nucleotide sequences coding for the protein sequence of the present invention under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe), high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC).

The term “mutant” in relation to the nucleotide sequence of the present invention means a variant of SEQ ID No. 1 but wherein that variant or the expression product thereof has the capability of substantially affecting the interaction of Gβ with a Cdc24p obtainable from C. albicans or a homologue thereof that is usually capable of being associated with the Cdc24p obtainable from C. albicans or the homologue thereof.

The term “mutant” in relation to the protein sequence of the present invention means a variant of SEQ ID No. 2 but wherein that variant has the capability of substantially affecting the interaction of Gβ with a Cdc24p obtainable from C. albicans or a homologue thereof that is usually capable of being associated with the Cdc24p obtainable from C. albicans or the homologue thereof.

The term “growth behaviour” includes growth per se (but not vegetative growth of yeast), growth control and growth orientation of cells. In some aspects, it includes at least growth orientation of cells. The term may also include the mating pattern (e.g. mating per se or mating behaviour) of cells.

For a preferred aspect of the present invention, any one or more of the nucleotide sequence of the present invention or the expression product thereof, or the mutant nucleotide sequence of the present invention or the expression product thereof, or the protein of the present invention, or the mutant protein of the present invention may be within a transgenic organism or cell (such as being an integral part thereof)—that is an organism or cell that is not a naturally occurring organism or cell and wherein the organism or cell has been prepared by use of recombinant DNA techniques. The transgenic cell may be part of or contained within tissue.

Preferably, the transgenic organism or cell is a yeast, an animal (such as a mammal) or an animal cell (such as a mammalian cell).

In preferred embodiments, the transgenic organism is a transgenic yeast or a transgenic mouse.

Transgenic yeast may be prepared by appropriately adapting the teachings of Ito et al Journal of Bacterioloy 153 163-168; Rose et al 1991 Methods in yeast genetics: a laboratory course manual Cold Spring Harbor, N.Y.: Cold Spring Harbor Press).

Transgenic mammals or mammalian cells may be prepared by appropriately adapting the teachings of Ausubel et al 1992 Short Protocols in Molecular Biology 2nd Ed. New York: John Wiley and Sons).

The transgenic organism or transgenic cell of the present invention therefore provides a simple assay system that can be used to determine whether one or more agents (e.g. compounds or compositions) have one or more beneficial properties. By way of example, the assay system of the present invention may utilise a mating phenotype and/or the assay system may be a two-hybrid interaction assay.

By way of example, if the transgenic organism is a transgenic yeast which comprises the nucleotide sequence presented as SEQ ID No. 1 or the expression product thereof (namely the protein sequence presented as SEQ ID No. 2) then the yeast could be used to screen for agents that bind to this nucleotide sequence or the expression product thereof and in doing so affect the growth behaviour of the yeast. If an agent produces such a detrimental effect (such as drastically reducing the ability of the yeast to mate), then that agent may also affect the interaction of Gβ with a Cdc24p obtainable from C. albicans or another Cdc24p entity that is usually capable of being associated therewith. This aspect of the present invention could allow workers to screen for anti-fungal agents, such as agents that could be used to treat or combat Candida.

By way of further example, if the transgenic organism is a transgenic yeast which comprises the nucleotide sequence presented as SEQ ID No. 1 or the expression product thereof then the yeast could be used to screen for agents that bind to this nucleotide sequence or expression product thereof and in doing so affect the growth behaviour of the yeast. If an agent produces a detrimental affect (such as drastically reducing the ability of the yeast to mate), then that agent is likely to detrimentally affect the interaction of Gβ with a homologue of C. albicans Cdc24p with which it is usually capable of being associated.

By way of further example, if the transgenic organism is a transgenic yeast which comprises a mutant of the nucleotide sequence in accordance with the present invention then the yeast could be used to screen for agents that affect the growth behaviour of the yeast.

By way of fiber example, if the transgenic organism is a transgenic yeast which comprises a homologue of the nucleotide sequence shown as SEQ ID No. 1 or an expression product thereof then workers could see if that homologue or the expression product thereof had an effect on the growth behaviour of yeast, and thus also to see if it had an effect on the interaction of Gβ with a homologue of the Cdc24p obtainable from C. albicans. In addition, workers could use those transgenic yeast to screen for agents that modified the effect—such as enhance the growth behaviour or detrimentally affect the growth behaviour. In this aspect, agents that affect the growth behaviour could have potential as anti-fungal agents.

The assays of the present invention may also be used to screen for agents that affect the interaction of a Cdc24p obtainable from C. albicans or a homologue of a Cdc24p obtainable from C. albicans with Gβ to determine whether that effect has a downstream effect on a Rho-family GTPase.

For example, with the present invention—such as by use of the assays of the present invention—it is possible to devise and/or to screen for peptide inhibitors which block GEF/Gβ interaction. In this regard, peptides and peptidyl derivatives based regions encompassing mutants may be used to block and/or antagonise a GEF obtainable from C. albicans Gβ interaction. Derivatives of these peptides (including peptide mimics) which bind with higher affinity may also be used. The perturbation of these interactions may be of therapeutic value for example in treatment of fungal disorders.

In addition, by use of the present invention it is possible to devise simple yeast based assay systems (utilising mating function and interaction reporters). These assay system will be extremely useful for high through-put screening to identify molecules perturbing a GEF/Gβ interaction wherein the GEP is obtainable from C. albicans or is a homologue thereof.

In addition, it is possible to devise and/or screen for agents that can modulate (e.g. interact), preferably selectively modulate (interact), with and affect Cdc24p/Gβ interactions wherein the Cdc24p is obtainable from C. albicans or is a homologue thereof. Hence, it would be possible to devise and/or to screen for anti-fungal agents directed at invasive and/or pathogenic yeasts such as, but not limited to Candida albicans and/or Cryptococcus neoformans and/or Aspergillus species such as Aspergillus niger.

If the assay of the present invention utilises a transgenic organism according to the present invention then transgenic organism may comprise nucleotide sequences etc. that are additional to the nucleotide sequences of the present invention in order to maintain the viability of the transgenic organism.

In accordance with a preferred aspect of the present invention, the nucleotide sequence is obtainable from, or the protein is expressable from the nucleotide sequence contained within, the respective deposit. By way of example, the respective nucleotide sequence may be isolated from the respective deposit by use of appropriate restriction enzymes or by use of PCR techniques.

The present invention will now be described only by way of example, in which reference is made to the following Figures:

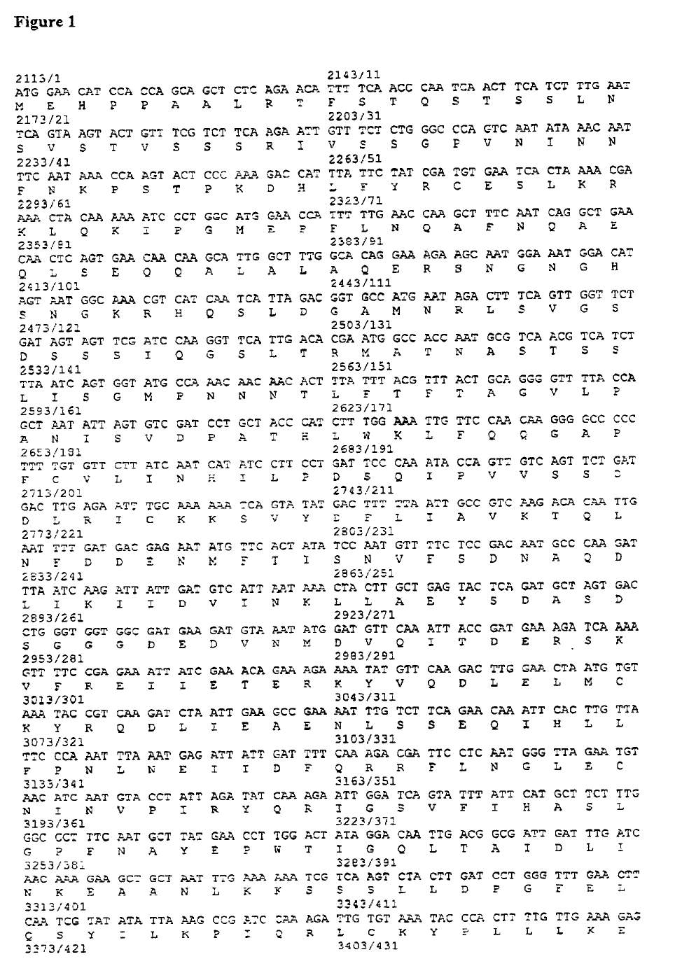

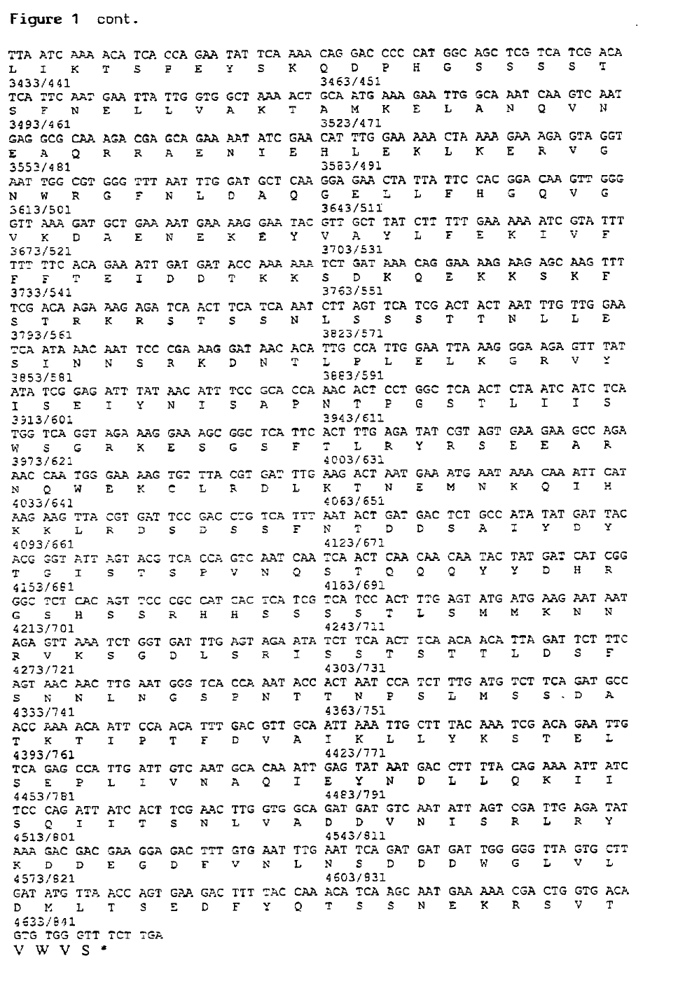

FIG. 1 which presents the nucleotide sequence (SEQ ID No 1) and the translated protein sequence (SEQ ID No 2) obtainable from C. albicans;

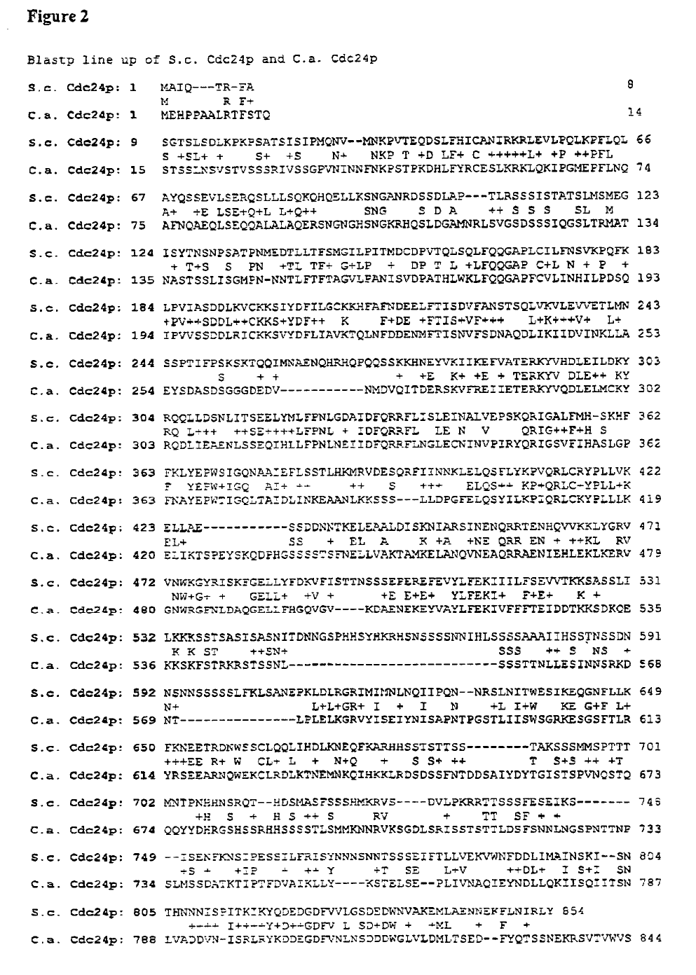

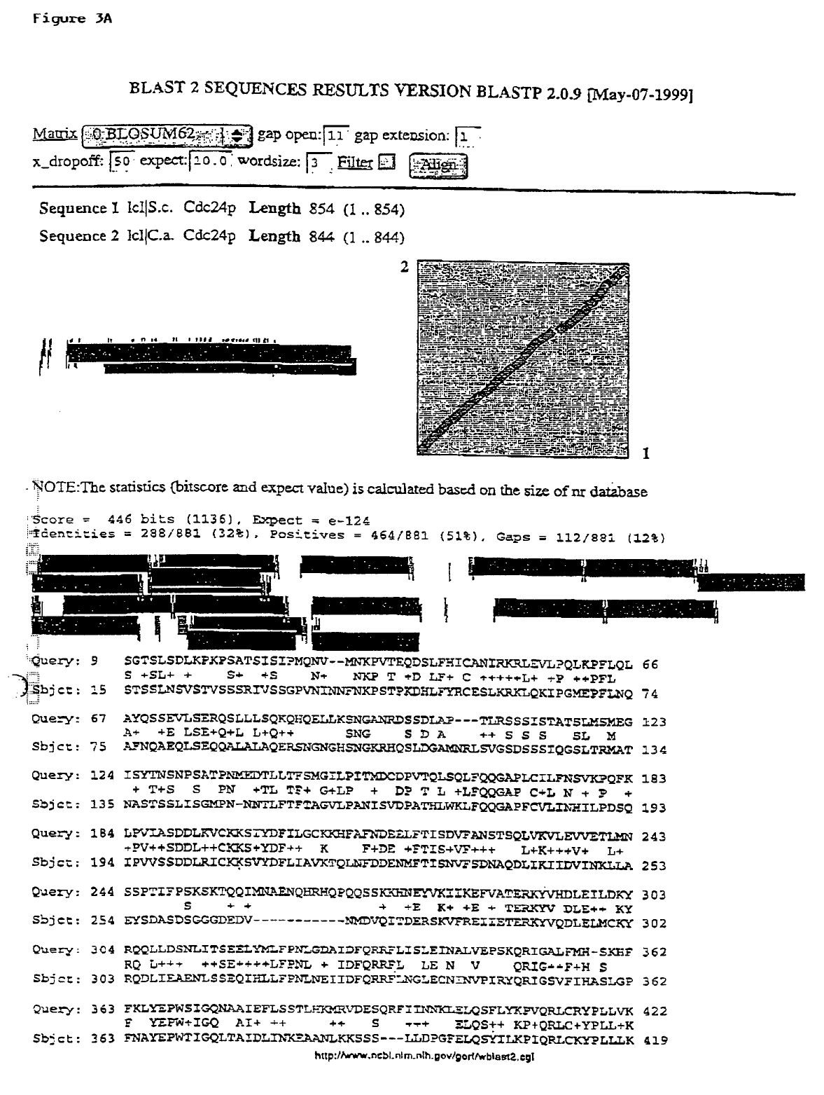

FIG. 2 which presents a BLAST line up of S. cerevisiae Cdc24p and C. albicans Cdc24p.

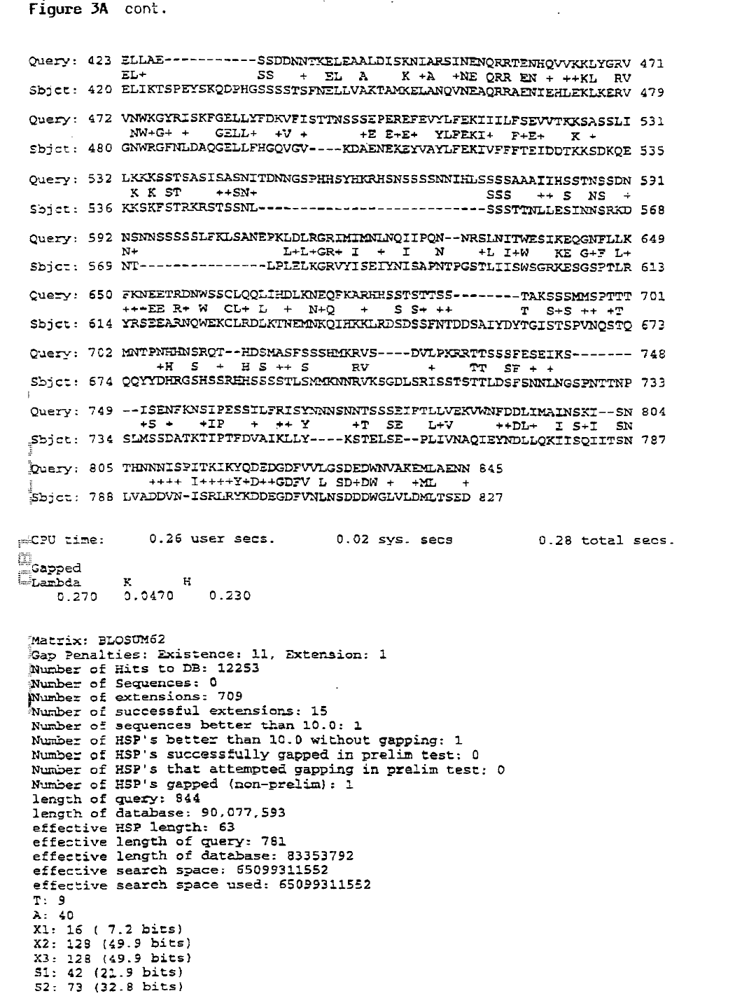

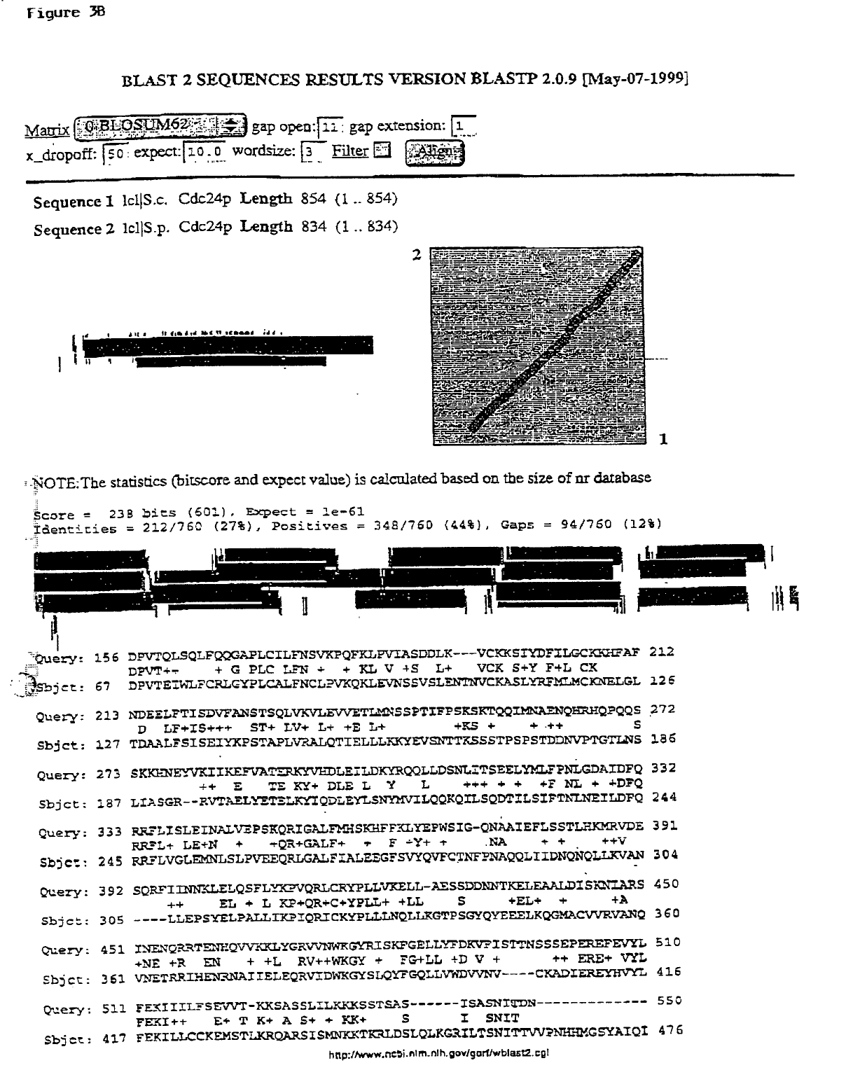

FIG. 3a shows the percent similarity and percent homology for a BLAST line up of S. cerevisiae Cdc24p and C. albicans Cdc24p;

FIG. 3b shows the percent similarity and percent homology for a BLAST line up of S. cerevisiae Cdc24p and S. pombe Cdc24p; and

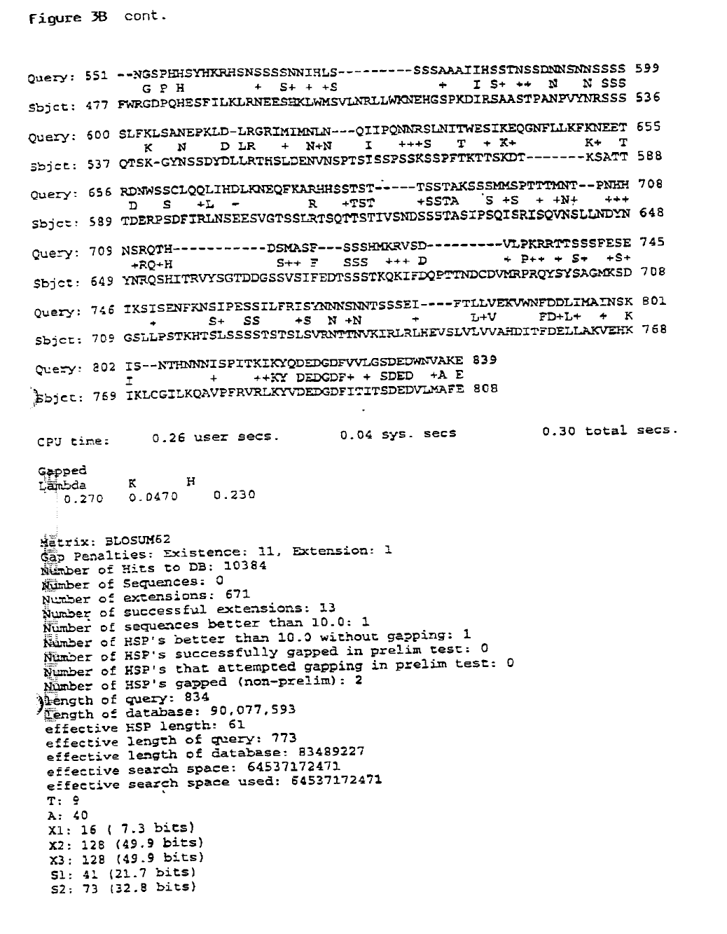

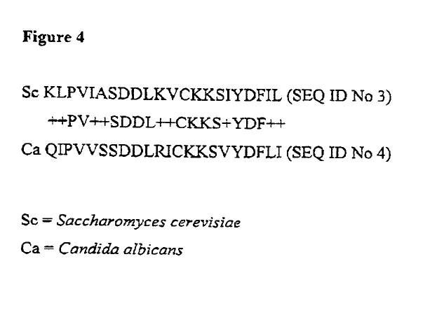

FIG. 4 which presents a comparison of the critical region (SEQ ID No 3) of S. cerevisiae Cdc24p with the corresponding sequence (SEQ ID No 4) in the C. albicans Cdc24p.

The Figures are discussed in more detail later on.

Materials and Methods

The C. albicans gene encoding the CDC24 protein was cloned in Bluescript as a 5.162 Kb genomic DNA fragment from KpnI to NsiI into KpnI Pstl of Bluescript. This cloned C. albicans CDC24 includes 1.95 Kb upstream of the CDC24 ATG start codon and 0.683 Kb downstream of TGA stop codon.

Degenerate primers were based on sequence similarities between S. cerevisiae and K. lactis CDC24 (the latter gene which we have cloned).

Forward primer:

```
5′-AAA TAC ATG CAA GAC TTA GA-3′
                G T G T G T G
                                C
                                T
```

Reverse primer:

```
5′- AAT TTT CTC AAA AAA ATA-3′
            G T G G G G
                                          C
                                          T
```

C. albicans CDC24 was initially identified using the above degenerate primers and PCR (polyrnerase chain reaction) from a C. albicans genomic DNA library in a 2 micron S. cerevisiae URA3 vector (Liu, H. P., Kohler, J., and Fink, G. R. [1994]. Suppression of hyphal formation in Candida albicans by mutation of a STE12 homolog. Science 266, 1723-1726). This primers were used to screen this library first as a pool of DNA in which we tried several degenerate primer pairs and finally on single bacterial library transformants. A process of serial dilution was used to identify a library clone that contained a piece of C. albicans CDC24 (with sequence that matched the degenerate primers). The clone was then sequenced and we used several different exact match C. albicans CDC24 primers based upon this sequence to get the entire gene. This was done in two steps, first the amino-terminal half including promoter and upstream region was cloned using one exact primer from the sequence we had and a primer to the library vector sequence. We isolated this clone by serial dilution screened by PCR and isolation of the library plasmid containing the C. albicans CDC24 insert. The second carboxyl-terminal half was isolated by PCR using one exact primer and a primer to the library vector sequence. After optimization of annealing temperatures the PCR product was cloned and three independent clones were sequenced. The two halves of C. albicans CDC24 were then combined by conventional cloning to produce the pBluescript C. albicans CDC24 clone I mentioned before.

Complementation Studies

The function of C. albicans CDC24 is tested by putting it in a S. cerevisiae yeast vector (using for example a 2 micron vector with a triose phosphate isomerase promoter from S. cerevisiae to drive expression) and looking for complementation of different S. cerevisiae cdc24 temperature sensitive mutants and also cdc24-m mating mutants in S. cerevisiae. A recent paper that has tested and shown functionality of a C. albicans gene in S. cerevisiae is: R. S. Care; J. Trevethick; K. M. Binley; and Sudbery, P. E. (1999). The MET3 promoter: a new tool for Candida albicans molecular genetics, Molecular Microbiology 34, 792-798.

### DISCUSSION

Cdc24p belongs to a diverse family of GEFs which include many mammalian proto-oncogenes². This group of proteins shares a conserved region consisting of a Dbl-domain (named after the human proto-oncogene Dbl) followed by a pleckstrin-homology domain (PH).

We have sequenced the entire CDC24 gene including promoter and terminator regions from C. aibicans. Sequence comparison between a Cdc24p obtainable from S. cerevisiae and C. albicans show about 32% identity and 51% similarity using a conventional BLAST line up. In particular, a comparison between the critical regions in the Cdc24p obtainable from S. cgerevisiae (as identified in WO 99/18213) and the corresponding region in the Cdc24p obtainable from C. albicans indicated that of 22 aimno acids, 13 were identical (59% identity) and 7 were similiar (32%).

The Cdc24p obtainable from C. albicans may provide a similar connection between G-protein coupled receptor activation and polarised cell growth as the Cdc24p from S. cerevisiae.

Hence, in accordance with the present invention there are provided the following uses and utilities of a Cdc24p interaction wherein the Cdc24p is obtainable from C. albicans. These include but are not limited to interactions with C. albicans and Gβ.

1) Peptide inhibitors which block GEP-Gβ activities/interaction wherein the GEF is obtainable from C. albicans or is a homologue thereof. Derivatives of these peptides (including peptide mimics) which bind with higher affinity may also be used. The perturbation of these interactions may be of therapeutic value for example in treatment of fungal disorders.

2) Simple yeast based assays systems (utilising mating function and interaction reporters) may be extremely useful for high through-put screening to identify molecules perturbing the GEF/Gβ activities/interaction wherein the GEF is obtainable from C. albicans or is a homologue thereof. In particular, the qualitative effect of a C. albicans GEF in S. cerevisiae mating will be amenable to large scale screening for anti-fungal agents.

3) Similar Cdc24p interactions such as Gβ interactions wherein the Cdc24p is obtainable from C. albicans may be ideal targets for anti-fungal drugs directed at the pathogenic yeast Candida.

### SUMMARY

1) We have sequenced the entire CDC24 gene including promoter and terminator regions obtainable from C. albicans. The C. albicans Cdc24p is a protein essential for viability and the life and growth of yeasts such as those obtainable from Candida species such as C. albicans. A sequence comparison between the Cdc24p obtainable from S. cerevisiae and C. albicans show about 32% identity and 51% similarity using a conventional BLAST line up.

2) We have already identified an important interaction been two general cellular components, Cdc24p and Gβ which provides a connection between G protein coupled receptor activation and polarised cell growth (see WO 99/18213). This work has been exemplified by work done with yeast genes/proteins, however, both cellular components involved have homologues in humans. We believe that the Cdc24p obtainable from C. albicans may provide an appropriate target for inhibition of cell growth.

3) In addition, we have identified sequences in the Cdc24p obtainable from S. cerevisiae (as identified in WO 99/18213) which are required for the interaction between two general cellular components, Cdc24p and Gβ, and which provide a connection between G protein coupled receptor activation and polarised cell growth. Specifically, we have identified a short stretch of one protein (Cdc24p) encompassing 75 aa sufficient for this interaction and three amino acid changes (within this stretch) which block the interaction and have physiological consequences.

4) A sequence comparison between the sequences which are required for the interaction between two general cellular components, Cdc24p and Gβ, in the Cdc24p obtainable from S. cerevisiae (as identified in WO 99/18213 and as outlined above) and the corresponding region in the C. albicans Cdc24p indicated that of 22 amino acids, 13 were identical (59% identify) and 7 were similiar (32%).

5) We propose that C. albicans Cdc24p interactions will have broad cellular ramifications and manipulation of these interactions (such as peptidic inhibitors and peptides mimicking activated species) may be of therapeutic value in anti-fungal treatments.

6) In addition, simple yeast based assays systems could be extremely useful for high throughput screening to identify molecules perturbing this interaction. In particular, a qualitative assay using a yeast mutant with a mating defect may prove useful in the design of agents, such as anti-fungal agents.

7) We also believe C. albicans Cdc24p GEF interactions may be an ideal target for anti-fungal drugs directed at invasive and pathogenic yeasts such as Candida albicans and Cryprococcus neoformans and Aspergillus niger.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### REFERENCES

1. Machesky, L. M. & Hall, A. Rho: a connection between membrane receptor signaling and the cytoskeleton. Trends In Cell Biolog 6, 304-310 (1996).

2. Cerione, R. A. & Zheng. Y. The Dbl family of oncogenes. Current Opinion In Cell Biology 8 216-222 (1996).

3. Whiteway, M., et al. The STE4 and STE18 genes of yeast encode potential b-subunits and γ-subunits of the mating factor receptor-coupled G protein. Cell 56, 467-477 (1989).

4. Sprague, G. F. J. & Thorner, J. W. Pheromone response and signal transduction during the mating process of Saccharomyces cerevisiae. In The molecular and cellular biology of the yeast Saccharomyces (eds. Jones E. W., Pringle, J. R. & Broach, J. R.) 657-744 (Cold Spring Harbor Press, Cold Spring Harbor, N.Y., 1992).

5. Leberer, E.. Thomas. D. Y. & Whiteway, M. Pheromone signalling and polarized morphogenesis in yeast. Current Opinion in Genetics & Developmnent 7, 59-66 (1997).

6. Simon, M. -N., et al. Role for the Rho-family GTPase Cdc42 in yeast mating pheromone signal pathway. Nature 376, 702-705 (1995).

7. Zhao, Z. S., Leung, T., Manser, E. & Lim, L. Pheromone signalling in Saccharomyces cerevisiae requires the small GTP-binding protein Cdc42p and its activator CDC24. Mol Cell Biol 15, 5246-57 (1995).

8. Sloat, B. F., Adams, A. & Pringle. J. R. Roles of the CDC24 gene product in cellular morphogenesis during the Saccharomyces cerevisiae cell cycle. J Cell Biol 89, 395-405 (1981).

9. Adams, A. E., Johnson. D. I., Longnccker, R. M., Sloat, B. F. & Pringle, J. R. CDC42 and CDC43, two additional genes involved in budding and the establishment of cell polarity in the yeast Saccharomyces cerevisiae. J Cell Biol 111, 131-42 (1990).

10. Chenevert, J., Valtz, N. & Herskowitz. I. Identification of genes required for normal pheromone-induced cell polarization in Saccharomyces cerevisiae. Genetics 136, 1287-96 (1994).

11. Trueheart J., Boeke, J. D. & Fink. G. R. Two genes required for cell fusion during yeast conjugation: evidence for a pheromone-induced surface protein. Mol Cell Biol 7, 2316-2328 (1987).

12. Segall, J. E. Polarization of yeast cells in spatial gradients of a mating factor, Proc Natl Acad Sci USA 90, 8332-6(1993).

13. Dorer, R., Pryciak, P. M. & Hartwell, L. H. Saccharomyces cerevisiae cells execute a default pathway to select a mate in the absence of pheromone gradients. J Cell Biol 131, 845-61 (1995).

14. Valtz, N., Peter, M. & Herskowitz. I. FAR1 is required for oriented polarization of yeast cells in response to mating pheromones. J Cell Biol 131, 863-73 (1995).

15. Zheng, Y., Cerione, R & Bender, A. Control of the yeast bud-site assembly GTPase Cdc42. Catalysis of guanine nucleotide exchange by Cdc24 and stimulation of GTPase activity by Bem3. J. Biol. Chem 269, 2369-72 (1994).

16. Zheng, Y., Bender, A. & Cerione, R. A. Interactions among proteins involved in bud-site selection and bud-site assembly in Saccharomyces cerevisiae. J Biol Chem 270, 626-30 (1995).

17. Chenevert, J., Corrado. K., Bender, A., Pringle, J. & Herskowitz, I. A yeast gene (BEMI) necessary for cell polarization whose product contains two SH3 domains. Nature 356, 77-9 (1992).

18. Hirschman, J. E., DeZutter, G. S., Simonds, W. F. & Jenness, D. D. The Gβγ complex of the yeast pheromone response pathway—subcellular fractionation and protein-protein interactions. J Biol Chem 272, 240-248 (1997).

19. Peter, M., Neiman, A. M., Park, H. O., Van Lohuizen, M. & Herskowitz, I. Functional analysis of the interaction between the small GTP binding protein Cdc42 and the Ste20 protein kinase in yeast. Embo Journal 15, 7046-7059 (1996).

20. Leberer, E., et al. Functional characterization of the Cdc42p binding domain of yeast Ste20p protein kinase. Embo Journal 16, 83-97 (1997)

21. Rose, M. D., Winston, F. & Hieter, P. Methods in yeast generics: a laboratory course manual (Cold Spring Harbor Press, Cold Spring Harbor, N.Y., 1991).

22. Miyamoto, S., et al. A DBL-homologous region of the yeast CLS4/CDC24 gene product is important for Ca(2+)-modulated bud assembly. Biochem Biophys Res Commun 181, 604-10 (1991).

23. Mitchell, D. A., Marshall, T. K. & Deschenes, R. J. Vectors for the inducible overexpression of glutahione-S-transferase fusion proteins in yeast. Yeast 9, 715-722 (1993).

24. Wash, A., Brachat, A., Alberti-Segui, C., Rebischung, C. & Philippsen, P. Heterologous HIS3 marker and GFP reporter modules for PCR-targeting in Saccharomyces cerevisiae. Yeast 13. 1065-1075 (1997).

25. Fromant, M., Blanquet, S. & Plateau, P. Direct random mutagenesis of gene-sized DNA fragments using polymerase chain-reaction. Analytical Biochemistry 224, 347-353 (1995).

26. Sprague, G. F. Assay of yeast mating reaction. Methods In Enzymol 194, 77-93 (1991).

27. Adams. A. E. & Pringle, J. R. Staining of actin with fluorochrome-conjugated phalloidin. Methods Enzymol 194, 729-31(1991).

28. Pringle, J. R. Staining of bud scars and other cell wall chitin with calcofluor. Methods Enzymol 194, 732-5 (1991).

29. Langle-Ronault, F. & Jacobs, E. A method for perfoming precise alterations in the yeast genome using a recyclable selectable marker. Nucleic Acids Research 23, 3079-3081 (1995).

30. James, P., Halladay, J. & Craig, E. A. Genomic libraries and a host strain designed for highly efficient two-hybrid selection in yeast. Genetics 144, 1425-1436 (1996).

101. Chang, E. C., Barr, M., Wang, Y., Jung, V., Xu, H. P., and Wigler, M. H. 1994. Cooperative interaction of S. pombe proteins required for mating and morphogenesis. Cell. 79: 131-41.

102. Pasteris, N. G., Cadle, A, Logie, L. J., Porteous, M., Schwartz, C. E., Stevenson, R. E., Glover, T. W., Wilroy, R. S., and Gorski. J. L. 1994. Isolation and characterization of the faciogenital dysplasia (aarskog-scott syndrome) gene—a putative Rho/rac guanine-nucleotide exchange factor. Cell. 79. 669-678.

103. Katzav, S., Martinzanca, D., and Barbacid, M. 1989 Vav. a novel human oncogene derived from a locus ubiquitously expressed in hematopoietic-cells. Embo Journal. 8: 2283-2290.

104. Miki, T., Smith. C. L., Long, J. E., Eva A., and Fleming, T. P. 1993. Oncogene ect2 is related to regulators of small GTP-binding proteins. Nature. 462-465.

105. Chan, A. M., McGovern, E. S., Catalano, G., Fleming T. P., and Miki, T. 1994. Expression cDNA cloning of a novel oncogene with sequence similarity to regulators of small GTP-binding proteins. Oncogene. 9: 1057-63.

106. Eva, A. and Aaronson, S. A. 1985. Isolation of a new human oncogene from a diffuse B-cell lymphoma Nature. 316: 273-275.

107. Ron, D., Tronick, S. R., Aaronson, S. A., and Eva, A. 1988. Molecular-cloning and characterization of the human dbl proto- oncogene—evidence that its overexpression is sufficient to transform nih/3t3 cells. Embo Journal. 7: 2465-2473.

108. Hart, M. J., Sharma, S., Elmasry, N., Qiu, R. G., Mccabe, P., Polakis, P., and Bollag, G. 1996. Identification of a novel guanine-nucleotide exchange factor for The Rho-gtpase. Journal Of Biological Chemistry. 271: 25452-25458.

109. Whitehead, I., Kiryk, H., Tognon, C., Trigo-Gonzalez, G., and Kay, R. 1995. Expression cloning of lfc, a novel oncogene with structural similarities to guanine nucleotide exchange factors and to the regulatory region of protein kinase C. J Biol Chem. 270: 18388-95.

110. Horii, Y., Beeler. J. F., Sakaguchi, K., Tachibana, M., and Miki, T. 1994. A novel oncogene, ost, encodes a guanine nucleotide exchange factor that potentially links Rho and Rac signaling pathways. Embo J. 13: 4776-86.

111. Glaven, J. A., Whitehead, I. P., Nomanbhoy, T., Kay, R., and Cerione, R. A. 1996. Lfc and lsc oncoproteins represent 2 new guanine-nucleotide exchange factors for the Rho-gtp-binding protein. Journal Of Biological Chemistry. 271. 27374-27381.

112. Toksoz, D. and Williams. D. A. 1994. Novel human oncogene ibc detected by transfection with distinct homology regions to signal-transduction products. Oncogene. 621-628.

113. Chan, A., Takai, S., Yamalda, K., and Mild, T. 1996. Isolation of a novel oncogene, netl, from neuroepithelioma cells by expression cdna cloning. Oncogene. 12: 1259-1266.

114. Hariharan, I. K. and Adams, J. M. 1987. Cdna sequence for human-bcr, the gene that translocates to the abl- oncogene in chronic myeloid-leukemia. Embo Journal. 6: 115-119.

115. Diekmann, D., Brill, S., Garrett, M. D., Totty, N., Hsuan, J., Monfries, C., Hall, C., Lim, L., and Hall, A. 1991. Bcr encodes a gtpase-activating protein for p21rac. Nature 651: 400-402.

116. Habets, G., Vanderkammen, R. A., Stam, J. C., Michiels, F., and Collard, J. G. 1995. Sequence of the human invasion-inducing tiaml gene, its conservation in evolution and its expression in tumor-cell lines of different tissue origin. Oncogene 1371-1376.

117. Sone, M., Hoshino, M., Suzuki, E., Kuroda, S., Kaibuchi, K., Nakagoshi, H., Saigo, K., Nabeshima, Y., and Hama. C. 1997. Still life, a protein in synaptic terminals of Drosophila homologous to GDP-GTP exchangers. Science. 275: 543-547.

What is claimed is:

1. An isolated polynucleotide comprising SEQ ID NO: 1 or a derivative, fragment, variant, or homologue thereof, wherein the expression product does not substantially affect the interaction of Gβ with Cdc24p or a homologue thereof.

2. The isolated polynucleotide of claim 1 wherein said polynucleotide is SEQ ID NO:1.

3. An isolated polynucleotide comprising a mutant of SEQ ID NO:1 or a derivative, fragment or homologue thereof, wherein the expression product is capable of reducing the interaction of Gβ with Cdc24p or a homologue thereof.

4. A vector comprising the isolated polynucleotide of claim 3.

5. The vector of claim 4 wherein said vector is a plasmid or a retrovirus.

6. A cell transformed by the vector of claim 4.

7. A method of inhibiting the growth of cells in a mammal comprising:
administering the vector of claim 4 in an amount effective to reduce the growth of said cells.

8. An isolated polypeptide comprising SEQ ID NO:2 or a derivative, fragment, variant or homologue thereof.

9. An isolated polypeptide encoded by the polynucleotide of claim 1.

10. An isolated polypeptide encoded by the polynucleotide of claim 3.

11. A method for identifying agents capable of affecting the interaction between Cdc24p or a homologue thereof with a Gβ or an associated Rho-family GTPase, comprising:
contacting an agent with a polypeptide according to claim 8;
determining whether the agent affects the interaction of the protein and Gβ or a Rho-family GTPase; and
identifying the agent as one which affects the growth behavior of cells if the agent affects the interaction of said polypeptide and the Gβ or Rho-family GTPase.

12. A pharmaceutical composition comprising, the isolated polypeptide of claim 10 in an amount effective to inhibit the growth of cells together with a pharmaceutically effective carrier.

13. A pharmaceutical composition comprising, the isolated polypeptide of claim 10 in an amount effective to induce the growth of cells together with a pharmaceutically effective carrier.

14. A kit comprising the nucleotide sequence of claim 1 or the expression product thereof and a Gβ polypeptide which interacts with Cdc24p or a homologue thereof.

15. A kit comprising the nucleotide sequence of claim 1 or the expression product thereof and a Gβ polypeptide which interacts with Cdc24p or a homologue thereof.

16. A kit comprising the polypeptide of claim 9 and a Gβ polypeptide which interacts with Cdc24p or a homologue thereof.

17. A kit comprising the polypeptide of claim 10 and a Gβ polypeptide which interacts with Cdc24p or a homologue thereof.

18. A method of inducing the growth of cells in a mammal comprising:
administering the polypeptide of claim 9 in an amount effective to reduce the growth of said cells.

19. The pharmaceutical composition of claim 12 wherein said pharmaceutical composition is an anti-fungal.

20. The polypeptide of claim 3 wherein said polypeptide is SEQ ID NO:4.
